(19) United States
(12) Patent Application Publication
(10) Pub. No.: US 2006/0051323 A1
(43) Pub. Date: Mar. 9, 2006

(54) Formulations comprising vitamin b12, method of production and use thereof

(76) Inventors: Hendrik Louis Bijl, Vlaardingen (NL); Sandjai Sardjoepersad, Den Haag (NL)

Correspondence Address: NIXON & VANDERHYE, PC, 901 NORTH GLEBE ROAD, 11TH FLOOR, ARLINGTON, VA, 22203, US

(21) Appl. No.: 10/534,390

(22) PCT Filed: Nov. 13, 2003
(86) PCT No.: PCT/EP03/12701

§ 371 Date: Jun. 2, 2005

(30) Foreign Application Priority Data
Nov. 14, 2002 (EP) 02102580.4

(51) Int. Cl. A61K 31/714; A61K 35/74

(52) U.S. Cl. 424/93.4; 514/52

(57) ABSTRACT

Particles comprising a vitamin B12-containing microbial biomass and a solid carrier and compositions comprising said particles and a method for the production of such particles and compositions.

### FIELD OF THE INVENTION

The present invention relates to particles comprising a vitamin B12-containing microbial biomass and to compositions comprising the same. The invention further relates to a method for producing such particles and compositions comprising said particles. The invention also relates to animal feed, human food or food supplements comprising said particles.

### BACKGROUND OF THE INVENTION

Microorganisms are known as valuable sources of a varied range of useful compounds. Several of these compounds are located either inside or are associated with the microbial cell. Generally, to recover such compounds after fermentation of the microorganisms, it is necessary to separate the compound from the microbial biomass. However, often such compounds are unstable to isolation techniques or when the used microorganisms are microbiologically safe and food-grade, the compounds are not produced in isolated form but are produced in dry formulation together with the biomass of the organism in which they are produced. Such formulations are especially suited for use as animal feed supplement. By “microbial biomass” we mean a microorganism-containing product resulting from fermentation, which consists of whole, preferably non-viable cells (i.e. dead or killed) and/or cell debris (e.g. broken/disintegrated/lysed cell walls).

Vitamin B12 is an important compound for humans and animals and it is an important animal feed supplement as growth enhancer. The term “vitamin B12” is used to describe compounds of the cobalt corrinoid family, in particular those of the cobalamin group. In this specification the term “vitamin B12” means all the cobalt corrinoids of the cobalamin group, which include in particular cyanocobalamin, hydroxocobalamin, methylcobalamin, 5′-adenosylcobalamin and 5′-desoxyadenosylcobalamin characterised by cyano, hydroxyl, methyl or 5′-desoxyadenosyl radical(s) respectively. The methylcobalamin and 5′-desoxyadenosylcobalamin compounds are known to be unstable to light in isolated form and are easily degraded to hydroxocobalamin in aqueous solution. For this reason, commercial vitamin B12 preparations consist of the more stable cyanocobalamin.

Vitamin B12 is often obtained in industrial fermentation methods using microorganisms known to produce vitamin B12.

A suitable method for the production of vitamin B12 via fermentation is described in International Patent Application WO00/37699. This document describes a non-continuous fermentation method for the production of vitamin B12 wherein a strain of Propionibacterium is cultured in two different fermentors under anaerobic and aerobic conditions respectively in a “fill and draw” fashion. The inhibiting effect of propionic acid on growth of Propionibacterium in the anaerobic phase can be considerably reduced, leading to increased biomass and increased vitamin B12 production at the end of the fermentation.

International Patent Application WO98/06868 describes a method for the preparation of compositions comprising vitamin B12 in a concentration (based on dry matter) higher than 0.1% w/w. Such compositions are produced by a method wherein microbial cells are cultured to (intracellularly) produce vitamin B12, after which the cells are partially lysed and/or damaged to cause release of vitamin B12 into the medium. After separation of microbial biomass from the vitamin B12-comprising liquid phase, and concentration of the latter, the resulting concentrate solution and the microbial biomass can be combined in different ratios and the resulting mixture(s) spray-dried. By this method, a spray-dried biomass can be obtained with a high concentration of vitamin B12.

Even though the production of biomass with a high concentration of vitamin B12 is advantageous in several ways (ease of transportation and consequent reduction of costs) a high concentration in vitamin B12 can be less desirable in some applications.

Spray-dried biomass can for example be used in animal feed. Prior to use in the production of feed, a lowering of the vitamin B12 concentration in a biomass with high concentration of vitamin B12 may be necessary. The latter is especially desirable when lower dosages of vitamin B12 in the animal diet (e.g. for poultry) are required. A possible solution to this problem could be to blend the spray-dried biomass with a solid carrier in order to reduce the vitamin B12 concentration prior to mixing with other feed components. The blends of vitamin B12-containing biomass and solid carrier could be added to other feed components, either directly or in the form of a premix, which also contains other vitamins, minerals and/or bioactive ingredients, in order to produce the final feed. In order to assure good distribution of vitamin B12 in the final feed compositions, especially when low dosages need to be applied, it is very important that the blends of solid carrier and vitamin B12-containing biomass are homogeneous.

Unfortunately, the inventors have observed that blends obtained by mixing spray-dried biomass and solid carriers are usually inhomogeneous. In addition, they are generally electrostatic, dusty and not free-flowing. Some of these problems can cause problems during handling of such blends on an industrial scale. Free-flowing characteristics could be increased by addition of inorganic solid carriers like silica, but this does not improve homogeneity of the blends. Moreover, some inorganic solid carriers, like silica, may be dusty, hazardous especially if inhaled, and not very desirable ingredients for animal feed. Lack of homogeneity of these blends is undesirable as it can lead to inaccurate dosage of the vitamin B12 into the final premix and/or feed, with unequal distribution of the nutrient between different animals. The latter is especially disadvantageous for smaller animals like poultry. Thus there is a need for improved vitamin B12 formulations, in particular for use in animal feed.

### DESCRIPTION OF THE INVENTION

Accordingly the present invention is concerned with providing improved formulations of vitamin B12-containing microbial biomass and a solid carrier where the above-mentioned problems can be at least mitigated, if not overcome.

The present inventors have found that when vitamin B12-containing microbial biomass and a solid carrier are present in the same particle, blending of vitamin B12-containing biomass with a solid carrier prior to mixing with the other feed components can become superfluous, and some of the problems related to the prior art blends can be overcome.

Therefore a first aspect of the invention provides a particle comprising a vitamin B12-containing microbial biomass and a solid carrier.

Particle Structures

The particles of the invention may have (3 or more) different morphologies (or structures). For instance, one possible morphology of the particle may be one in which the solid carrier is mainly concentrated near the centre of the particle while the vitamin B12-containing microbial biomass constitutes a sort of continuous film of coating material around it. This is the preferred structure. The particle may have a core or central portion comprising the solid carrier and a coating (or outer layer) comprising the biomass.

A second possibility is the reverse of the first. It may be one in which distribution of respectively vitamin B12-containing microbial biomass and solid carrier is reversed, i.e. the biomass is concentrated in the centre of the particle while the solid particles are more on the outside. Thus the particle may have a core or central position comprising the biomass and a coating, or outer layer comprising the carrier.

A third possibility may be one in which the particle is actually constituted by a matrix of vitamin B12-containing microbial biomass in which particles of solid carrier are entrapped or vice-versa.

Particle Sizes

The size of the particles may vary, being preferably from 0.2 μm to 2000 μm. The particle size may be from 10 μm to 1000 μm, preferably comprised between 20 μm to 500 μm, even more preferably from 50 μm to 300 μm, most preferably from 50 μm to 150 μm.

Preferably, the particles of the invention have a (substantially) homogeneous particle size distribution. The phase “homogeneous particle size distribution” is intended to mean that the overall particle size distribution can be relatively narrow, such that at least 70% w/w of the particles, preferably at least 80% w/w, and more preferably 90% w/w of the particles have a particle size comprised between 20 and 500 μm, more preferably comprised between 50 μm and 300 μm.

The particles of the invention may have a vitamin B12 concentration of typically about 0.05%-5% w/w, more typically 0.1 to 4% w/w, 0.1 to 3% w/w, 0.1 to 2% w/w or 0.1-1% w/w, usually not exceeding 10% w/w.

Typically the moisture content of the particles is comprised between 5-10% w/w such as 6 to 8% w/w or 7 to 10% w/w.

The invention further provides a composition comprising particles according to the invention.

The particles, or a composition (essentially consisting) of particles according to the invention may have a (substantially) homogeneous distribution of vitamin B12 on or in the solid carrier. The particles may be free flowing and/or not dusty. It may be (substantially) non-electrostatic (for example, they may not stick to glass). The particles can be produced very economically.

The “vitamin B12-containing microbial biomass” is generally a micro-organism-containing-product resulting from fermentation of microorganisms capable of producing vitamin B12 and cultured under conditions conducive thereof. It can mean a biomass (either alive or dead) comprising cells that comprise vitamin B12, such as cells that produce (or have produced) vitamin B12. The micro-organism-containing-product consists of preferably non-viable (e.g. dead), whole (or intact) cells and/or cell debris comprising vitamin B12.

Vitamin B12-containing (or producing cells), or microbial biomass, preferably comprises a bacterial strain, such as of the genus Acetobacterium, Acetobacter, Agrobacterium, Alcaligenes, Arthrobacter, Azobacter, Bacillus, Clostridium, Corynebacterium, Escherichia, Eubacterium, Flavobacterium, Methanobacillum, Methanosarcina, Mycobacterium, Propionibacterium, Proteus, Pseudomonas, Rhizobium, Rhodopseudomonas, Salmonella, Serratia, Streptococcus, Streptomyces or Xanthomonas. Preferably a bacterium is used which is safe for consumption by humans, e.g. GRAS, and/or animals. In particular, the bacterium preferably does not produce endo- or exotoxins. Propionibacteria in particular are often food-grade and satisfy these criteria.

Therefore in a preferred embodiment of the invention the particle is characterised in that the vitamin B12-containing microbial biomass is from the genus Propionibacterium.

Preferred Propionibacterium species used at this regard are P. freundenreichii, P. theonii, P. jensenii, P. shermanii and P. acidipropionici. In one embodiment it is preferred that a single bacterial strain is present.

Solid Carrier

The solid carrier used in the particles of the invention may be a particulate material or powder which is preferably non-hygroscopic. The carrier can be suitable for use in a spray-drying, multi-stage and/or fluid bed drying techniques. The carrier may be edible or digestible (either by animals and/or humans). The carrier will exclude cells or parts thereof.

The solid carrier in the particles according to the invention preferably has low bulk density. This may allow them to be used in the above-mentioned drying techniques. Preferably the density is comprised between 400 and 1200 kg/m3, preferably between 400 and 1000 kg/m3, more preferably about 500 kg/m3. Preferably the particle size of the carrier is equal to or lower than 500 μm, preferably equal to or lower than 300 μm, generally comprised between 10-300 μm, more preferably comprised between 10-200 μm, most preferably comprised between 30-150 μm.

Generally the solid carrier has a moisture content of 2-15% w/w a powder for example, 5 to 10% w/w such as 6, 8, 12 or 14% w/w.

Preferably the solid carrier comprises a carbohydrate, a protein, or a mixture thereof. Suitable solid carriers comprise (powders of) casein, whey, milk, maltodextrin, corn steep solids, starch, edible flour, or mixtures thereof. Most preferably the solid carrier comprises edible flour. With “edible flour” it is intended to cover a finely ground meal (essentially consisting of starch and protein) obtainable from edible cereal grains or seeds (e.g. wheat, rice, maize, barley, oat, rye, etcetera), from legumes (e.g. beans, peas, etcetera) or from edible tubers or fruits such as potatoes or bananas, or a mixture thereof. Edible flour can have the advantage of being cheap, light and of being a desirable component in animal feed.

In one embodiment it is preferred that the solid carrier is not a (e.g. crystallisable) sugar such as lactose, saccharose, dextrin or other maltodextrin. In a preferred embodiment, the particles of the invention are characterised in that the weight ratio between the vitamin B12-containing microbial biomass and the solid carrier is between 0.2-5, preferably between 0.25-4 or 0.3-3, more preferably between 0.5-2.

The particles according to the invention can be produced according to any method suitable to the formation of composite particles, like spray-drying, fluid bed drying, multi-stage drying. Spray-drying, fluid bed drying and multi-stage drying techniques are known to those skilled in the art.

Spray-drying

Preferably the particles of the invention are produced by spray-drying or multi-stage drying techniques. For example, a liquid (suspension of) vitamin B12-containing microbial biomass can be spray-dried in the presence of a solid carrier (preferably in powder form). The terms spray-drying or spray-dryer are used in a broad sense, to cover both pure spray-drying or spray dryer and multi-stage drying or multi-stage dryer.

Accordingly the invention in a second aspect provides a method for the production of particles comprising vitamin B12-containing microbial biomass and a solid carrier. At its broadest, the method of the second aspect comprises co-spray drying the biomass and solid carrier. In other words, the biomass and solid carrier are both spray dried simultaneously, and preferably in contact with one another.

The spray drying of both of the biomass and the solid carrier can result in the particles of the first aspect. Preferably, the particles will comprise a central or core portion of the sold carrier, and a coating or outer layer of the biomass. The biomass and solid carrier will preferably be supplied to the spray-dryer in separate streams, or through different ports or inlets. Suitably, the solid carrier and biomass are only (and first) mixed once inside the spray-dryer.

A solid carrier may be in solid form, such as a powder, or in a liquid form, which is a slurry. The biomass is preferably in a liquid form, preferably a liquid suspension. The biomass (such as in the form of a liquid) may have been subjected to several processing steps prior to being co-spray dried with the solid carrier. It may have been subjected to concentration and/or evaporation, dia filtration and/or pasteurisation. Thus, one or more of these steps may have been performed on a liquid comprising the biomass before spray drying occurs. Preferably, inside the spray dryer, the biomass is atomised. Atomisation may occur before the biomass is mixed with the solid carrier.

In a preferred method, a liquid comprising (e.g. a suspension of) vitamin B12-containing microbial biomass and a solid carrier (e.g. in powder form) are conveyed into (for example, a drying chamber) of a spray-dryer, preferably in or through separate streams or ports. The liquid (suspension) and the solid carrier can then come into contact with each other inside the spray-dryer chamber.

Fermentation and Pre-Spray Drying Steps

The vitamin B12-containing microbial biomass used in the method of the invention is preferably obtainable in or from an industrial fermentation process using microorganisms which produce vitamin B12. These include bacteria belonging to the bacterial strains mentioned above. Several fermentation methods suitable to the microbial production of vitamin B12 are known to those skilled in the art. Preferably, the vitamin B12-containing microbial biomass is obtainable from a bacterial strain of the genus Propionibacterium. Several methods are known in the art for the fermentation of Propionibacterium strains under conditions conducive to the production of vitamin B12. An example is described in International Patent Application WO00/37699.

Generally the microbial cells containing vitamin B12 are concentrated and optionally purified at the end of the fermentation by one or more methods suitable to this purpose (e.g. evaporation, ultrafiltration, diafiltration, etc.).

Preferably a concentration (based on dry matter) of microbial biomass in the liquid suspension is obtained which allows economical spray-drying operation. In a preferred embodiment the liquid (suspension of) vitamin B12-containing microbial biomass used in the method according to the invention has a concentration of 50-300 g/l, preferably 100-300 g/l, more preferably 200-300 g/l or 250-300 g/l based on dry mass per litre of liquid (i.e. concentrate).

Optionally it is possible to use a vitamin B12-containing microbial biomass with a relatively low concentration, for example of about 120-150 g/l, and further concentrate the liquid suspension, for example up to about 200-300 g/l, 220-300 g/l or 250-300 g/l, just prior to spray-drying. A concentrator/evaporator positioned upstream to the spray-dryer can be used for this purpose. Thus, a fermentation broth can be subjected to concentration and/or evaporation to form the liquid to be co-spray dried with the solid carrier.

Acids

It is known that carboxylic acids with a low molecular weight like acetic and propionic acid are produced during fermentation of Propionibacterium strains. The latter can pose a problem as the presence of acids during spray-drying of the corresponding microbial biomass can cause difficulties in the drying process and can cause stickiness of the end product. Therefore, the liquid (e.g. suspension of Propionibacterium microbial) biomass is preferably treated, prior to spray-drying or multi stage drying, for example by diafiltration. This may reduce the acid concentration to a desirable value.

Optionally the vitamin B12-containing microbial biomass is pasteurised prior to spray-drying.

Spray-dryer

The method for the production of the particles according to the invention can be performed on a conventional spray-dryer or multi stage dryer. This type of equipment is generally used in many applications, e.g. in the dairy industry.

Generally a spray-dryer (or multi-stage dryer) comprises at least a drying chamber, such as with a distribution element for atomising a liquid to be spray-dried. It may also have means for supplying (drying) gas and/or means for discharging the (spray-dried) product from the device. Besides the above-mentioned elements, a multi stage-dryer further comprises one or more fluidised beds. A spray-dryer or multi-stage dryer suitable for use in the method according to the invention may further comprise means suitable to supply the solid carrier into the drying chamber.

Therefore a spray-dryer (or multi-stage dryer) suitable for use in the method of the invention can comprise at least two (product)-inlet ports, generally positioned on the upper part of the spray-dryer chamber. Through one inlet port the liquid (suspension of) microbial biomass can be atomised and conveyed into the spray-dryer chamber. Said inlet port is furnished with means suitable to atomise the liquid such as an atomiser (e.g. nozzle, rotating disk atomisers etc.). A second product-inlet port can be used to convey the solid carrier, (generally in powder form) into the drying chamber. A slurry of the carrier can also be applicable. Optionally the spray-dryer may comprise means for the recovery of fine particles. Said (fine) particles can be reintroduced into the drying chamber by means of a third product-inlet port or into the pipeline conveying either the microbial biomass or the solid carrier into the system or dyer. Optionally, the spray-dryer is part of a multi-stage dryer comprising one or more fluidised beds.

Typically an inlet temperature of the air in the drying chamber of the spray-dryer is used which is between 120-250° C., preferably between 160-220° C. such as 180 to 200° C. The outlet temperature of the air is generally comprised between 60-95° C. for example 60-90° C. or 70-80° C.

Both streams of solid carrier and (atomised) liquid (suspension of) microbial biomass are conveyed into the drying chamber. A stream of droplets can be produced by atomisation of the liquid (suspension of microbial biomass). The (e.g. atomised) liquid may then be brought into contact with the solid carrier (e.g. in powder form). This usually happens inside the drying chamber. One can then produce particles comprising vitamin B12-containing microbial biomass and the solid carrier.

Ratio of Biomass to Carrier

The method of the invention advantageously allows adjustment of the amount of vitamin B12-containing microbial biomass and/or (on) the solid carrier, in order to assure an optimal distribution of vitamin B12 containing biomass on the particle. Advantageously the invention also allows adjustment of the amount of vitamin B12 on the solid carrier, depending both on the content of vitamin B12 in the microbial biomass and on the final application of the resulting particles.

Preferably the weight ratio between the vitamin B12-containing microbial biomass and the solid carrier used in a method of the invention is between 0.2-5, preferably between 0.25-4 or 0.3-3, more preferably between 0.5-2.

The method can be used to produce particles having the properties described above. In a preferred embodiment the invention relates to particles obtainable by the method. Such particles can have a number of advantages compared to e.g. particles formed by mixing spray-dried biomass and solid carrier, or formed by spray drying a mixture of biomass and solid carrier. For example, the particles generally have a homogeneous mean particle size distribution. The particles usually have visual homogeneity i.e. no separation can usually be observed between microbial biomass and solid carrier. Particles obtained in this way are also generally less hygroscopic, free flowing, less dusty and/or more free of moulds and bacterias. The particles are particularly useful when a lower concentration of vitamin B12 is desirable e.g. for use in animal feeds.

One advantage related to the method of the invention is that particles with a homogeneous mean particle size distribution can be obtained. Another advantage in the method according to the invention is that the spray-drying step allows production of “pasteurised” compositions comprising the particles according to the invention. This is especially advantageous when certain types of solid carrier, which are not always free of microorganisms and yeast (e.g. edible flour) are used.

The invention thus provides particles comprising a vitamin B12-containing microbial biomass and a solid carrier obtainable by a method of the invention. Said particles have the desirable characteristics already described above. The invention in a third aspect provides compositions comprising the particles of the first aspect or particles preparable by the second aspect. Preferred features and/or characteristics of one aspect of the invention are applicable to another aspect mutatis mutandis.

Animal Feed

The particles of the invention can be used as or in the production of animal feed. To this end, the particles containing vitamin B12 are added to other feed components, either directly or in the form of a premix, which may also contain other vitamins, enzymes, minerals and/or bioactive ingredients.

Therefore the invention also provides an animal feed comprising particles according to the invention.

Feeding an animal a diet comprising a feed according to the invention promotes its growth.

Thus the invention also provides the use of an animal feed according to the invention to promote the growth of an animal.

A further aspect of the invention relates to a premix or additive composition to be added to one or more edible feed substance(s) or ingredient(s), for example to prepare (or for supplementation of) a feed composition. This can comprise the particles of the fist aspect or preparable by the method of the second aspect. The premix can be “diluted” by a factor of 10 to 1,000 (so that the premix constitutes 10% to 0.1% of final feed) when making the animal feed. This premix may be in the form of granules or pellets.

The invention also relates to a process for the preparation of an animal feed composition, the process comprising adding to (or supplementing) an animal feed, or to one or more edible feed substance(s) or ingredient(s), the particles of the invention.

Another aspect of the invention relates to a process for promoting growth, feed conversion or antibacterial activity, in a monogastric or non-ruminant animal, the process comprising feeding the animal particles of the invention.

Suitable animals include farm, monogastric and/or non-ruminant animals such as pigs (or piglets), poultry (such as chickens and turkeys), calves, veal calves or aquatic (e.g. marine) animals, for example fish.

The compositions of the invention, in particular additive or premix compositions, can be either in liquid or solid form. If a solid, then this may be a powder, a granulate, extrudate or it may be pellets. For a solid form, the amount of water present may be below 20, 15 or even 10%, such as from 2 to 10%, 3 to 8% or 4 to 7%.

The remainder may comprise carbohydrates and/or carbohydrate polymers (such as starch and/or modified starch), for example at least 70, 80, 90 or 95%, such as from 75 to 90%. The composition may have a coating, for example if it is in a pellet, granulate, or extrudate form. There may thus be one or more coats on the outside of the composition, comprising one or more coating materials. If present, the coating (or coating materials) may be present at from 1 to 10%, such as from 2 to 6%, optimally at from 3 to 5%. The composition may have one or more stabilisers (such as glycerol and/or sorbitol) and/or one or more preservatives (such as sorbate and/or benzoate).

If the composition is a liquid, then the water (or moisture) content will be higher. The water content may be up to 40, 50 or 60%, for example from 25 to 65%, optimally from 35 to 55%. If a stabiliser is present, this may be at an amount of from 45 to 65%, such as from 50 to 60%, optimally from 52 to 58%. The stabiliser is preferably sorbitol and/or glycerol.

A description of the preparation of pellets and granules, in particular carbohydrate based enzyme granulates, is described in WO-A-98/54980 (International Application No. PCT/EP98/03327). This and all other documents mentioned have their contents incorporated herein by reference.

The composition may comprise a carrier which may comprise at least 15% of an edible carbohydrate polymer. The carrier may be in particulate or powder form. However, if the composition is a liquid, it may be in the form of a solution or a slurry. The polymer preferably comprises glucose, or glucose-containing units, although it can contain glucopyranose units, amylose and/or amylopeptin. In addition, or instead of starch, a glucan, peptin or glycogen can be used. Preferably at least 15%, such as at least 30%, at least 40%, for example at least 60%, optimally at least 80% of the composition (or the solid carrier) comprises the carbohydrate polymer.

Additional details of enzyme-containing compositions for animal feed can be found in WO-A-98/55599 (International Application No. PCT/EP98/03328).

Animal feed compositions of the invention will usually contain one or more feed ingredients or substances. These are ingredients and substances intended for consumption by an animal, and is therefore in a form suitable for ingestion and nutrition for an animal. Preferably the feed composition is both edible and digestible by the animal.

Suitably the ingredients and/or substances have a dry matter content of at least 80, 85, 90 or 95%. The protein content of the composition (or the substances and/or ingredients) may vary considerably, but may be from 5 to 20%, such as 10 to 15%, for example vegetable and/or plant products or parts thereof, such as buckwheat, rice, wheat, barley or corn. Substances or ingredients with higher protein contents, such as from 45 to 95%, e.g. 50 to 80%, may be provided, for example peanuts, poultry feathers, soy bean (or products thereof), sunflower (e.g. seeds) or casein. Preferred animal feed compositions may therefore comprise one or more of oats, pea (seeds), peanuts, soy beans, sunflower, canola, casein, coconut, corn, meat, millet, potato, rice, safflower and/or wheat. Preferably the composition (and substances or ingredients) have a crude fibre content below 30%, 25%, 20%, 15% or even below 10%. Similarly, the calcium content may be below 2%, such as 1%, below 0.5% and preferably less than 0.2%. The total phosphorous content of the (animal feed composition) is preferably from 2 to 0.01%, such as from 1 to 0.1%, optimally less than 0.5%.

The precise substances and ingredients can vary depending on the animal to be fed. An alternative composition may comprise one or more of bakery waste, sugar beet, brewers grain, canola, cassaya, corn, fababean, fish (such as anchovy or herring meal), lentils, meat and/or millet.

The particles of the invention can also be used in the production of a human food, foodstuff or food, dietary or nutritional supplement or a pharmaceutical composition. Therefore the invention provides any of these compositions comprising particles according to the invention.

The invention will now be illustrated, by way of Examples, which are not intended to be limiting.

### EXAMPLES

General Methods

Fermentation broth from Propionibacterium freudenreichii CBS 929.97 was obtained as described in International Patent Application WO00/37699.

The fermentation broth was concentrated by means of ultrafiltration (on polysulfon MW cut off 5-10 kD, Koch HFK 151 VSV) or microfiltration (on Membralox ceramic 0.1 μm) up to a biomass concentration of 100-150 g/l.

After ultrafiltration or microfiltration, the propionic acid in the biomass had a concentration of about 25-30 g/l. To reduce the concentration of propionic and acetic acid in the biomass, the biomass concentrate was diafiltered with water. This diafiltration was performed by an in-line addition of water to the concentrate at the same rate as the permeate flow. The diafiltration was stopped at a propionic acid concentration lower than 5 g/l. At this purpose a ratio (v/v) water: concentrate of 3-4:1 was applied.

After diafiltration the concentrated biomass was pasteurised during 1 minute at a temperature of 90-940 C (either by direct steam injection or heating by a plate heat exchanger).

The pasteurized biomass was further concentrated by a multistage (vacuum) falling film evaporator with vapor recompression. This type of evaporator is known to those skilled in the art.

The following conditions were applied.

| Biomass feed rate | 2000-3000 | 1/h |
|---|---|---|
| | (corresponding to 300 kg dry matter/h) | |
| Pre-heater temperature | 920 | C. |
| 1st stage temperature | 65-700 | C. |
| 5th stage temperature | 50-550 | C. |
| Temperature of concentrate | 45-500 | C. |

| | |
|---|---|
| Biomass concentration | 22-26% (1250 kg/h) |

The biomass concentrate was spray-dried on a Multi Stage Dryer (NIRO AS, Denmark).

The following set up was used in all the experiments.

The vitamin B12-containing biomass was fed into the drying chamber by a nozzle with a biomass feed rate of 1250 kg dry matter/h).

| | | |
|---|---|---|
| Nozzle pressure | 190-195 | bar |
| Air inlet temperature (co current) | 200-2200 | C. |
| Air outlet temperature | 75-920 | C. |
| Air Internal fluid bed temperature | 55-600 | C. |
| Air 1st external fluid bed temperature | 30-350 | C. |
| Air 2nd external fluid bed temperature | 15-200 | C. |
| Powder temperature | <300 | C. |

Fines were returned via a cyclone to the nozzle area.

### (Comparative) Example 1

In this example vitamin B12-containing biomass was spray-dried in absence of solid carrier applying the above-mentioned spray-drying conditions.

### Example 2

In this example 600 kg of vitamin B12-containing spray-dried biomass obtained in Example 1 was mixed in an external powder mixer (batch) with 300 kg of wheat flour and 1 kg of silica (Aerosil 200®).

### Example 3

In this example 120 g MgSO4.7H2O per kg of concentrate was added to the diafiltered biomass concentrate (120 g/l biomass concentration) before evaporation. The mixture was evaporated to a dry matter content of 32% and spray dried as described above.

### Example 4

In this example vitamin B12-containing biomass was spray-dried in presence of wheat flour as a solid carrier applying the above-mentioned spray-drying conditions. The wheat flour was dosed as a powder at a rate of 180-220 kg/h Both streams of solid carrier and atomised liquid suspension of microbial biomass were separately conveyed into the spray dryer chamber. The powder was dosed into the spray dryer chamber close to the area of the nozzle feed stream.

Results

The characteristics of the compositions comprising vitamin B12-containing spray-dried biomass obtained in Examples 1 to 4 were analysed and are reported in the following table.

| | Example | | | |
|---|---|---|---|---|
| | 1 | 2 | 3 | 4 |
| Vitamin B12 content (mg/kg) | 1600 | 1080 | 985 | 1110 |
| Dry matter (% w/w) | 94 | 94 | 86 | 94 |
| Presence of lumps | no | no | yes | no |
| Dust (mg/kg) | 50 | 10500 | 410 | 320 |
| Flowability | ok | ok | ok | ok |
| Particles (% w/w) with particle | n.r. | 91 | 99 | 82 |
| size lower than 300 μm) | | | | |
| Total plate count per g | 20 | 14000 | 20 | 500 |
| Moulds per gram | 100 | 500 | 10 | 20 |
| Visual homogeneity | yes | no | no | yes |

Visual homogeneity in the context of the present table means that the distribution of microbial biomass on the solid carrier is visually homogeneous, i.e. no separation is observed between spray-dried microbial biomass and solid carrier.

By comparing the results obtained in Example 4 with those obtained in the other examples it is clear that compositions essentially consisting of the particles of the invention can be homogeneous, not hygroscopic, free-flowing, not dusty and almost free of moulds and bacteria.

1. A particle comprising vitamin B 12-containing microbial biomass and a solid carrier.

2. A particle according to claim 1 wherein the weight ratio between the vitamin B 12-containing microbial biomass and the solid carrier is between 0.2-5 and/or the particles have been prepared by spray-drying (i.e. the particles are spray-dried).

3. A particle according to claim 1 wherein the (or average) particle size is comprised between 0.2 um and 2000 um, and/or particles have been prepared by co-spray drying the biomass and the solid carrier.

4. A particle according to claim 1 wherein the vitamin B 12-containing microbial biomass comprises a bacterial strain of the genus Acetobacterium, Acetobacter, Agrobacterium, Alcaligenes, Arthrobacter, Azobacter, Bacillus, Clostridium, Corynebacterium, Escherichia, Eubacterium, Flavobacterium, Methanobacillum, Methanosarcina, Mycobacterium, Propionibacterium, Proteus, Pseudomonas, Rhizobium, Rhodopseudomonas, Salmonella, Serratia, Streptococcus, Streptomyces and/or Xanthomonas.

5. A particle according to claim 1 wherein there is a core or central portion comprising the solid carrier and a coating or outer layer comprising the biomass.

6. A particle according to claim 1 wherein the solid carrier comprises a carbohydrate, a protein, or a mixture thereof.

7. A particle according to claim 6 wherein the solid carrier comprises casein, whey, milk, maltodextrin, corn steep solids, starch, edible flour, or a mixture thereof preferably as a powder.

8. A particle according to claim 7 wherein the edible flour is obtainable from wheat, rice, barley, maize, oat, rye, potato, banana, or a mixture thereof.

9. A method for producing particles comprising vitamin B 12-containing microbial biomass and a solid carrier, the method comprising co-spray drying the biomass and the carrier.

10. A method according to claim 9 wherein a liquid comprising the vitamin B 12-containing microbial biomass and a solid carrier in powder form are used.

11. A method according to claim 9 wherein the biomass and carrier are conveyed into a drying chamber of a spray-dryer through separate streams or ports.

12. A method according to claim 9 wherein a liquid suspension of biomass and the solid carrier are contacted with each other inside the spray-dryer.

13. A method according to claim 9 wherein the weight ratio between the vitamin B 12-containing microbial biomass and the solid carrier is between 0.2 and 5.

14. A method according to claim 9, wherein the solid carrier has a particle size equal to or lower than 500 pm.

15. A method according to claim 9 wherein the vitamin B12-containing microbial biomass comprises, or is obtainable from a bacterial strain of the genus Propionibacterium.

16. A method according to claim 9 wherein the solid carrier is a carbohydrate, a protein, or a mixture thereof.

17. A method according to claim 9 wherein the solid carrier comprises edible flour.

18. A method according to claim 9 wherein the liquid comprising the biomass has been subjected to concentration or evaporation, pasteurisation and/or diafiltration prior to spray drying and/or the liquid is atomised inside the spray dryer.

19. A method of claim 10 wherein the concentration of vitamin B12-containing microbial biomass in the liquid suspension is between 50 and 300 g/1.

20. A particle comprising vitamin B12-containing microbial biomass and a solid carrier obtainable by a method according to claim 9.

21. A composition comprising particles according to claim 1.

22. An animal feed comprising particles according to claim 1.

23. A composition edible by humans comprising particles according to claim 1.

24. A composition according to claim 23, which is a human food or food supplement, foodstuff, nutritional or dietary supplement or a pharmaceutical composition.
